(19) United States
(12) Patent Application Publication
(10) Pub. No.: US 2025/0325311 A1
(43) Pub. Date: Oct. 23, 2025

(54) AN IMPLANT FOR ORTHOPEDIC STABILIZATION

(71) Applicant: Ritesh Arjunkumar RATHI, Ahmedabad (IN)

(72) Inventor: Ritesh Arjunkumar RATHI, Ahmedabad (IN)

(21) Appl. No.: 19/121,128

(22) PCT Filed: Oct. 27, 2023
(86) PCT No.: PCT/IB2023/060849

§ 371 Date: Apr. 14, 2025

(30) Foreign Application Priority Data
Oct. 27, 2022 (IN) 202221061269

(51) Int. Cl. A61B 17/80; A61F 2/30

(52) U.S. Cl. CPC A61B 17/80; A61F 2/30771; A61F 2002/30433; A61F 2002/3065; A61F 2002/30654; A61F 2002/30785

(57) ABSTRACT

The present disclosure relates to an implant (100) for orthopedic stabilization. The implant (100) facilitates partial mobility between a pair of bones of a joint. The implant includes a first plate (10) on operative portion of first bone of the joint, a stem (15) extending from operative surface of first plate (10), a ball (42) formed on operative end of stem (15). A second plate (20) mounted on operative portion of second bone of the joint. The first cavity (44) configured on operative surface of second plate (20). An interconnecting plate (30) mounted on operative portion of second plate (20). The second cavity (45) configured on operative portion of interconnecting plate (30). A recess is formed by attaching second plate (20) and interconnecting plate (30). The recess configured to receive ball (42) therein facilitates partial mobility between the pair of bones in operative configuration of implant (100).

### FIELD

The present disclosure relates to the field of prosthetics (Implants) used for healing bone fractures.

### BACKGROUND

The background information herein below relates to the present disclosure but is not necessarily prior art.

Plates are widely used in orthopedics for the stabilization of various kinds of fractures to regain the function of the fracture. Generally, these plates are made from metal, i.e., titanium or stainless steel. Mostly these plates are straight. However, in recent times, pre-contoured plates matching the shape of the bone have been used. Despite significant attempts, the shape of the plate does not always match the contour of the bone. Due to a mismatch in the shape between bone and plate and bone being weaker than the plate, the bone has to bear the increased stress. This leads to increased stress concentration at the fracture location.

Additional stress at the fracture site is unfavorable for the healing of the bone fracture. Moreover, mismatches in the shape of the bone possess a technical challenge for the surgeon during the operation.

There is, therefore, felt a need for an implant for orthopedic stabilization that alleviates the aforementioned drawbacks.

### OBJECTS

Some of the objects of the present disclosure, which at least one embodiment herein satisfies, are as follows:

One object of the present disclosure is to provide an implant for orthopedic stabilization.

Another object of the present disclosure is to provide an implant for orthopedic stabilization that provide an increased degree of freedom.

Yet another object of the present disclosure is to provide articulating elements that accommodate complex contours of fractured bones.

Still another object of the present disclosure is to provide an implant for orthopedic stabilization that provides ease of assembly.

Yet another object of the present disclosure is to provide an implant for orthopedic stabilization that facilitate flexibility in the movement of the joint.

Still another object of the present disclosure is to provide an implant for orthopedic stabilization that provides ease of mounting with the bones.

Yet another object of the present disclosure is to provide an implant for orthopedic stabilization that restores the functionality of the joint.

Other objects and advantages of the present disclosure will be more apparent from the following description, which is not intended to limit the scope of the present disclosure.

### SUMMARY

The present disclosure envisages an implant for orthopedic stabilization. The implant is configured to facilitate partial mobility between a pair of bones of a joint. The implant comprises a first plate, a second plate, and an interconnecting plate. The first plate is configured to be mounted on an operative portion of the first bone of the joint. A stem of a predetermined length extends from the operative surface of the first plate. A ball of predefined diameter formed on an operative end of the stem. The second plate is configured to be mounted on an operative portion of a second bone of the joint. A first cavity is defined by an arcuate hollow space. The first cavity is configured on the operative surface of the second plate. The interconnecting plate is configured to be mounted on an operative portion of the second plate. A second cavity is defined by an arcuate hollow space. The second cavity is configured on at least one operative portion of the interconnecting plate. A recess is configured to be formed by attaching the second plate and the interconnecting plate by aligning the first cavity and the second cavity to define the recess for the ball.

In an aspect, the recess is configured to receive the ball therein to facilitate partial mobility between the pair of bones in an operative configuration of the implant.

In an aspect, the ball of the first plate is configured to be placed within the recess established between the second plate and the interconnecting plate that forms a ball and socket joint.

In an aspect, a first internal solid extrusion formed on the interconnecting plate that is configured to receive a fastening means.

In an aspect, a second internal solid extrusion is formed on the second plate that is configured to receive the fastening means. The fastening means originates from the first internal solid extrusion of the interconnecting plate that is configured to establish an interconnection between the second plate and the interconnecting plate.

In an aspect, the first plate includes first holes configured to facilitate precise positioning and fixation of the implant on the operative portion of the first bone of the joint.

In an aspect, the second plate includes second holes configured to facilitate precise positioning and fixation of the implant on the operative portion of the second bone of the joint.

In an aspect, the first plate is detachably attached to the second plate and the interconnecting plate.

In an aspect, the implant further includes an array of the first plates, the second plates, the interconnecting plates are configured to be arranged to form an implant chain to accommodate variable lengths of fractured bones or different orientation of curved non-linear bones of the joint.

In an aspect, the first plate is oriented at an angle of inclination to the interconnecting plate.

In an aspect, the plates are constructed from material selected from the group including metals, plastics, composites, bone substitutes or any combination thereof.

In an aspect, the plates are constructed from materials selected from the group including metals, plastics, composites, bone substitutes or any combination thereof.

### BRIEF DESCRIPTION OF THE ACCOMPANYING DRAWING

An implant for orthopedic stabilization, of the present disclosure, will now be described with the help of the accompanying drawing, in which:

FIG. 1 shows a perspective exploded isometric view of the implant, in accordance with an embodiment of the present disclosure;

FIG. 2a shows a perspective sectional view of FIG. 1;

FIG. 2b illustrates a perspective view of a First Plate in accordance with an embodiment of the present disclosure of FIG. 2a;

FIG. 3 shows a perspective sectional view of FIG. 1 in an assembled state of the implant;

FIG. 4 shows a perspective front view of a front plate in accordance with an embodiment of the present disclosure;

FIG. 5 shows a perspective top view of FIG. 3;

FIG. 6 shows a perspective isometric view of Implant chain, in another embodiment of the present disclosure;

FIG. 7 shows an isometric view of the implant with holes serving as fastening locations; and

FIG. 8 shows a side view of FIG. 7.

### LIST OF REFERENCE NUMERALS

100—Implant
200—Implant Chain
300—Implant with Holes
10, 10a, 10b—First Plate
20, 20a, 20b—Second Plate
30, 30a—Interconnecting plate
40, 40a—Ball and Socket Joint
42—Ball
44—First Cavity
45—Second Cavity
50, 50a—Fastening Means
60a, 60b—Holes
70—First internal solid extrusion
80—Second internal solid extrusion

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open-ended transitional phrases and therefore specify the presence of stated features, elements, modules, units, and/or components, but do not forbid the presence or addition of one or more other features, elements, components, and/or groups thereof. The particular order of steps disclosed in the method and process of the present disclosure is not to be construed as necessarily requiring their performance as described or illustrated. It is also to be understood that additional or alternative steps may be employed.

When an element is referred to as being “mounted on,” “engaged to,” “connected to,” or “coupled to” another element, it may be directly on, engaged, connected or coupled to the other element. As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Terms such as “inner,” “outer,” “beneath,” “below,” “lower,” “above,” “upper,” and the like, may be used in the present disclosure to describe relationships between different elements as depicted from the figures.

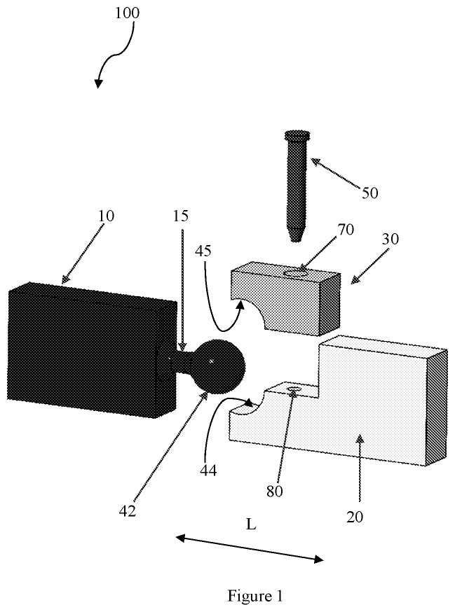

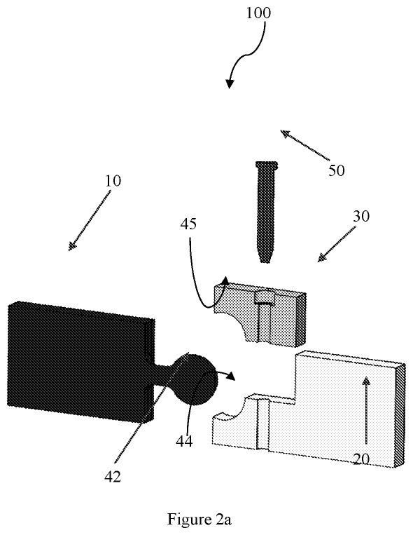

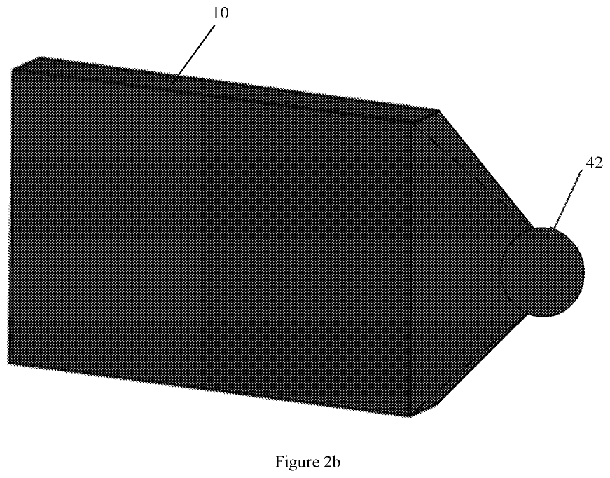

Referring to FIGS. 1 and FIG. 2a-2b, An implant 100 for orthopedic stabilization is illustrated to facilitate at least partial mobility between a pair of bones of a joint. In an aspect, the implant includes a first plate 10 which is configured to be mounted on an operative portion of a first bone of the joint. A stem 15 of a predetermined length extends from the operative surface of the first plate 10. A ball 42 of predefined diameter formed on an operative end of the stem 15.

The implant further includes a second plate 20 that is configured to be mounted on an operative portion of a second bone of the joint. A first cavity 44 is defined by an arcuate hollow space and is configured on the operative surface of the second plate 20.

In an embodiment, the ball 42 is directly attached to at least one operative side surface of the first plate 10 without the requirement of the stem 15. The side surface of the first plate is extended conically to accommodate the ball therein 42. FIG. 2b illustrates a perspective view of a First Plate in accordance with an embodiment of the present disclosure of FIG. 2a;

The implant further includes an interconnecting plate 30 is configured to be mounted on an operative portion of the second plate 20. The second cavity is defined by an arcuate hollow space and configured on at least one operative portion of the interconnecting plate 30.

Correspondingly, a recess is configured to be formed by attaching the second plate 20 and the interconnecting plate 30 by aligning the first cavity 44 and the second cavity 45 to define the recess for the ball 42. The recess is configured to receive the ball 42 therein to facilitate partial mobility between the pair of bones in an operative configuration of the implant 100.

In first embodiment, the ball 42 of the first plate 10 has a rough surface.

In second embodiment, the ball 42 of the first plate 10 has a smooth surface.

In an embodiment, the second cavity 45 of the second plate 20 has a rough surface or a smooth surface.

In an embodiment, the ball 42 may be configured on both operative sides of the first plate 10.

In an embodiment, a first internal solid extrusion 70 formed on the interconnecting plate 30 is configured to receive at least one fastening means 50.

In another embodiment, a second internal solid extrusion 80 formed on the second plate 20 is configured to receive the fastening means 50. The fastening means 50 originates from the first internal solid extrusion 70 of the interconnecting plate 30 to establish an interconnection between the second plate 20 and the interconnecting plate 30 to curtail relative motion.

In an embodiment, the second plate 20 and the interconnecting plate 30 are arranged such that the first internal solid extrusion 70 and the second internal solid extrusion 80 are in-line t receive the fastening means 50 therein.

In another embodiment, the first plate 10 is detachably attached to the second plate 20 and the interconnecting plate 30.

In another embodiment, the first plate 10 is oriented at an angle of inclination to the second plate 20 based on the orientation of the bones of the joint.

In another embodiment, the first plate 10 is oriented at an angle of inclination to the interconnecting plate 30.

In an embodiment, the stem is pivotally mounted within the periphery of the recess to thereby enable the movement of the bone of the joint.

Advantageously, the mounting of the interconnecting plate 30 on the second plate 20 facilitates ease of mounting of the stem 15 on the recess formed on the second plate. In addition, the recess formed on the second plate facilitates ease of movement of the implant within bone.

Further, the plates 10, 20, 30 are configured to be attached to a fractured bone for stabilizing the fractured bone and to enable the mobility between the bones of the joint.

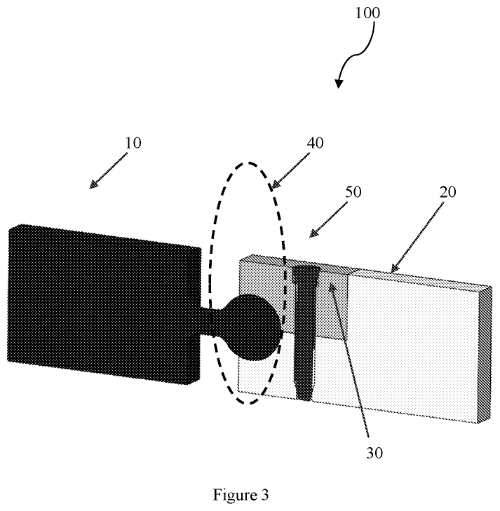

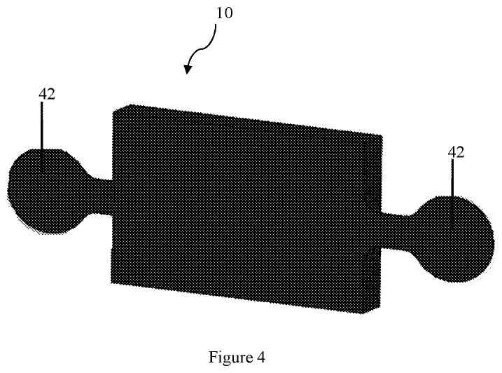

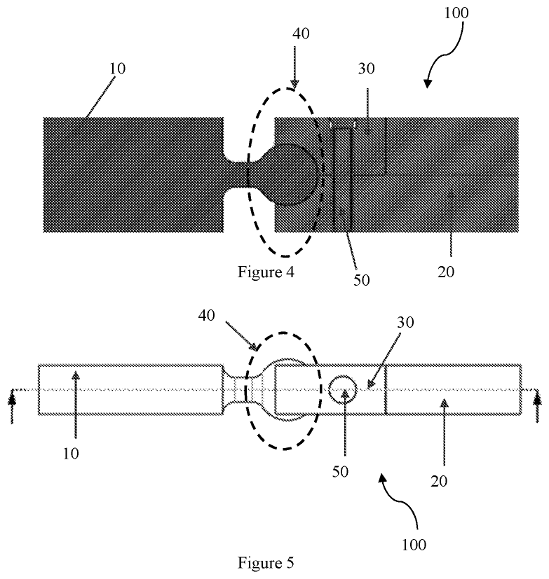

Referring to FIGS. 3, 4 and 5, the ball 42 of the first plate 10 is configured to be placed within the recess established between the second plate 20 and the interconnecting plate 30 that forms a ball and socket joint 40. The ball and the socket joint 40 are configured to accommodate angular mismatch between the positions of the plates 10, 20, 30 when attached to fractured bones. This facilitates accurately capturing the profile of the fractured bone being treated, as the accurate capturing of the fractured bone is desired to be positioned accurately.

Moreover, the fractured bone is stabilized after the implant 100 is attached to the fractured bone during the healing period of the fractured bone. Thus, the ball and socket joints 40, 40a offer flexibility in accommodating the contour of the fractured bone.

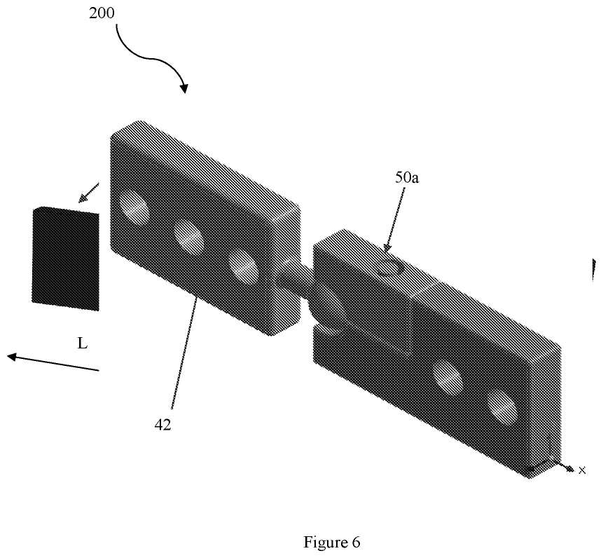

Referring to FIG. 6, the implant 100 further includes an array of the first plates, the second plates, and interconnecting plates 10, 10a, 20, 20a, 30, 30a are configured to be attached together to form an implant chain 200. The implant chain 200 thus formed, facilitates ease in accommodating variable lengths of fractured bones or different orientation of curved non-linear bones of the joint as desired to be treated. This further facilitates standardization of the plates 10, 20, 30. Further, the inventory is reduced.

In another embodiment, the ball 42 of the first plate 10 is configured to be placed within the recess established between the second plate 20, 20a and the interconnecting plate 30, 30a that forms a ball and socket joint 40, 40a.

In another embodiment, the operative longitudinal direction L of one implant 200 is oriented at an angle relative to the operative longitudinal direction L1 of another implant 100.

In another embodiment, the operative longitudinal direction L, L1 of each of array of the first plates (10, 10a) and the second plates (20, 20a) of the implants 100 are oriented in a parallel manner to each other.

In another embodiment, the plates 10, 10a, 10b, 20, 20a, 20b, 30, 30a of the implant chain 200 may have the same dimensions or different dimensions as desired.

In another embodiment, the length L, L1 of the plates 10, 10a, 10b, 20, 20a, 20b, 30, 30a of the implant chain 200 may be the same or different, as desired.

The plates 10, 10a, 10b, 20, 20a, 20b, 30, 30a are constructed from materials selected from the group including metals, plastics, composites, bone substitutes or any combination thereof.

In another embodiment, the first plate 10, the second plate 20 and interconnecting plate 30 are preferably fabricated from or coated with low-friction, wear and impact-resistant, biocompatible materials, Such as, for example, titanium, Stainless Steel, Surgical alloys, chrome molybdenum alloys, cobalt chromium alloy, Zirconium oxide ceramic, non-absorbable polymers and other anticipated bio-compatible metallic or polymeric materials.

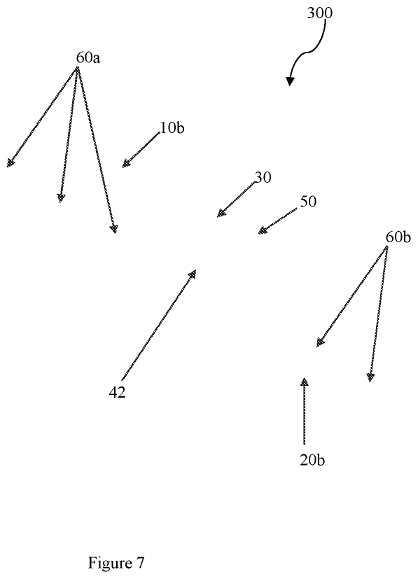

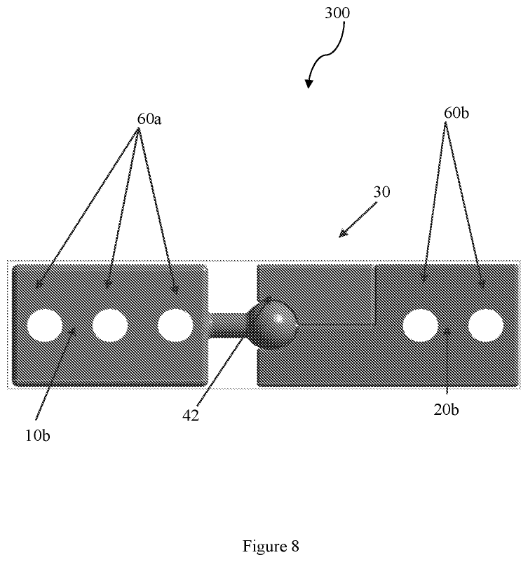

Referring to FIGS. 7, and 8, the implant with holes 300 to facilitate precise positioning and fixation of the implant 100. The first plate 10b includes first holes 60a configured to facilitate precise positioning and fixation of the implant 100 on the operative portion of the first bone of the joint.

In another embodiment, the second plate 20b includes second holes 60b configured to facilitate precise positioning and fixation of the implant 100 on the operative portion of the second bone of the joint.

The holes 60a, 60b of the first plate 10 and the second plate 20 facilitate positioning and fixation of the implant 300 to the bone as desired. Further, the hole 60 facilitates fastening locations for suitable attachment to the bone as desired.

During application, either of the implants 100, 200, 300 is configured to be attached to contoured bones having undulating surfaces.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

### TECHNICAL ADVANCES AND ECONOMIC SIGNIFICANCE

The present disclosure described herein above has several technical advantages including, but not limited to, the realization of an implant for orthopedic stabilization that:
facilitates accurate positioning of the fractured bone;
provides an increased degree of freedom;
accommodates complex contours of fractured bones;
reduces stress concentration in the fractured area of the bone;
facilitates flexibility in the movement of the joint;
provides ease of mounting with the bones;
restores the functionality of the joint; and
offers maneuverability in performing the fracture repair operation.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. An implant (100) for orthopedic stabilization, said implant configured to facilitate at least partial mobility between a pair of bones of a joint, said implant (100) comprising:
at least one first plate (10) configured to be mounted on an operative portion of a first bone of the joint;
a stem (15) of a predetermined length extending from at least one operative surface of said first plate (10);
a ball (42) of predefined diameter formed on an operative end of said stem (15);
at least one second plate (20) configured to be mounted on an operative portion of a second bone of the joint;
at least one first cavity (44) defined by an arcuate hollow space and configured on at least one operative surface of said second plate (20);
an interconnecting plate (30) configured to be mounted on an operative portion of said second plate (20);
at least one second cavity (45) defined by an arcuate hollow space and configured on at least one operative portion of said interconnecting plate (30); and
a recess configured to be formed by attaching said second plate (20) and said interconnecting plate (30) by aligning said first cavity (44) and said second cavity (45) to define said recess for said ball (42);
wherein said recess is configured to receive said ball (42) therein to facilitate at least partial mobility between the pair of bones in an operative configuration of said implant (100).

2. The implant (100) as claimed in claim 1, wherein said ball (42) of said first plate (10) is configured to be placed within said recess established between said second plate (20) and said interconnecting plate (30) that forms a ball and socket joint (40, 40a).

3. The implant (100) as claimed in claim 1, wherein a first internal solid extrusion (70) formed on said interconnecting plate (30) configured to receive at least one fastening means (50).

4. The implant (100) as claimed in claim 1, wherein a second internal solid extrusion (80) formed on said second plate (20) configured to receive said fastening means (50) originates from said first internal solid extrusion (70) of said interconnecting plate (30) that is configured to establish an interconnection between said second plate (20) and said interconnecting plate (30).

5. The implant (100) as claimed in claim 1, wherein said first plate (10) includes a plurality of first holes (60a) configured to facilitate precise positioning and fixation of said implant (100) on the operative portion of the first bone of the joint.

6. The implant (100) as claimed in claim 1, wherein said second plate (20) includes a plurality of second holes (60b) configured to facilitate precise positioning and fixation of the implant (100) on the operative portion of the second bone of the joint.

7. The implant (100) as claimed in claim 1, wherein said first plate (10) is detachably attached to said second plate (20) and said interconnecting plate (30).

8. The implant (100) as claimed in claim 1, wherein said implant (100) further includes an array of said first plates, said second plates, said interconnecting plates (10, 10a, 10b, 20, 20a, 20b, 30, 30a) and are configured to be arranged to form an implant chain (200) to accommodate variable lengths of fractured bones or different orientation of curved non-linear bones of the joint.

9. The implant (100) as claimed in claim 1, wherein said first plate (10) is oriented at an angle of inclination to said second plate (20) based on the orientation of the bones of the joint.

10. The implant (100) as claimed in claim 1, wherein said plates (10, 10a, 10b, 20, 20a, 20b, 30, 30a) are constructed from material selected from the group including metals, plastics, composites, bone substitutes or any combination thereof.
